(19) United States
(12) Patent Application Publication
(10) Pub. No.: US 2024/0148936 A1
(43) Pub. Date: May 9, 2024

(54) SCAFFOLD FOR TISSUE REGENERATION, PARTICULARLY FOR BONE REGENERATION, AND MANUFACTURING METHOD THEREOF

(71) Applicant: PHARMAEXCEED S.R.L., PAVIA (IT)

(72) Inventors: Ferdinando Auricchio, Pavia (IT); Elia Bari, Pavia (IT); Michele Conti, Pavia (IT); Sara Perteghella, Pavia (IT); Franca Scocozza, Pavia (IT); Marzio Sorlini, Pavia (IT); Maria Luisa Torre, Pavia (IT)

(73) Assignee: PHARMAEXCEED S.R.L., PAVIA (IT)

(21) Appl. No.: 18/549,434

(22) PCT Filed: Mar. 9, 2022
(86) PCT No.: PCT/IB2022/052098

§ 371 Date: Sep. 7, 2023

(30) Foreign Application Priority Data
Mar. 9, 2021 (IT) 102021000005441

(51) Int. Cl. A61L 27/36; A61L 27/26; A61L 27/52; A61L 27/54

(52) U.S. Cl. CPC A61L 27/3604; A61L 27/26; A61L 27/3687; A61L 27/3691; A61L 27/52; A61L 27/54; A61L 2300/30; A61L 2430/02

(57) ABSTRACT

A scaffold for tissue regeneration, particularly for bone regeneration, comprises a three-dimensional supporting structure made of a biocompatible and biodegradable material; the supporting structure is functionalized with secretome, in particular mesenchymal stem cell secretome, preferably in lyophilized form (lyosecretome).

### CROSS-REFERENCE TO RELATED APPLICATIONS

This patent application claims priority of Italian Patent Application No. 102021000005441 filed on Mar. 9, 2021, the entire disclosure of which is incorporated herein by reference.

### TECHNICAL FIELD

The present invention relates to a scaffold for tissue regeneration, particularly for bone regeneration in the field of regenerative medicine. In particular, the present invention falls within the context of tissue engineering as multidisciplinary field that applies the principles of engineering and biology to produce biological substitutes capable of restoring tissue, for example bone tissue, affected by a disease, a trauma or due to aging. More precisely, the technical field of the present invention refers to a scaffold (i.e. a framework, often three-dimensional, suitable for cell support) loaded (even only partly) with functional material derived from cells, the release of which promotes the regeneration of tissue, for example bone tissue, affected by a disease, a trauma or due to aging.

### STATE OF THE ART

In the orthopaedic field, surgical procedures to restore bone tissue are necessary in the case of loss of substance that can occur following a number of clinical situations, such as malformations, traumas and neoplasms. To date, the surgical procedures most commonly used to solve these problems consist in transplant or in the implantation of artificial prostheses; however said conventional treatments have considerable critical aspects, including the increase of the need for transplants compared with the number of donors, the need for immunosuppressive therapies to avoid rejection of the transplanted tissue and the impossibility of regrowth and regeneration of the damaged tissue in the case of implanted prostheses. A promising alternative to conventional approaches is represented by tissue engineering the purpose of which is to generate living tissue, including bone tissue, that is immunologically, functionally, structurally and mechanically similar to the native tissue to be replaced so as to obtain optimal integration with the surrounding tissue.

Tissue engineering provides a valid alternative and is based on the use of cell scaffolds to implant into the areas to be regenerated.

The term scaffold is meant as a support, often three-dimensional and often porous, produced with biocompatible and optionally biodegradable, material, having the ability to temporarily replace natural tissue promoting mechanisms of differentiation, adhesion and proliferation of the cells loaded thereon. During production of the extracellular matrix by the cells, the scaffold tends to biodegrade and, simultaneously, to be substituted by the regenerated biological tissue, thus restoring the compromised function and at the same time avoiding surgical reintervention to remove the prosthetic element.

Scaffolds are normally produced with two categories of materials: natural materials and synthetic materials. Polymers belong to the second category and those that are resorbable and most commonly used include polyglycolic acid (PGA), polylactic acid (PLA), poly(lactic-co-glycolic) acid (PLGA) and polycaprolactone (PCL). In detail, PCL is one of the most widely used biomaterials in the field of tissue engineering to produce systems to replace natural bone tissue. The widespread use of PCL is legitimized by suitable chemical, physical and mechanical properties and by confirmed qualities of biocompatibility and biodegradability; it is moreover a material approved by the United States FDA. Polymer scaffolds can be manufactured by means of a wide range of conventional techniques. The aforesaid conventional techniques have recently been augmented by new techniques based on additive manufacturing or three-dimensional printing (3D bio-printing) with which polymer scaffolds of specific and complex shape with properties of controlled porosity can be produced. Using 3D bio-printing technology, the scaffold is produced through the deposition of subsequent layers (layer-by-layer). Generally, the 3D bio-printing process starts with the definition of a digital model of the scaffold to be manufactured, which can be obtained, for example, by means of a CAD (computer-aided design) drawing, or directly from scanned images of the patient's damaged bone segment. A 3D model, to be produced through automatic image processing, is thus obtained. Subsequently, the 3D CAD model is converted into a series of instructions, generally in G-code, required to activate 3D printing.

The cells form, together with the scaffolds, the source of raw material for tissue engineering. Therefore, a further critical point for the engineering of a tissue is the choice of the correct cell source. The cells used can be of various type, for example: autologous, allogeneic, xenogeneic and stem cells (both embryonic and adult). Among adult stem cells, mesenchymal stem cells (MSCs) currently represent a great resource in tissue engineering. In fact, MSCs can be easily isolated from the patient, particularly from bone marrow or adipose tissue, and expanded in culture to a physiologically relevant number. Moreover, MSCs have the ability to differentiate in different cell lines, such as osteoblasts, chondrocytes, miocytes and adipocytes, giving rise to different cell tissues, such as bone, cartilage, tendon, muscle and adipose tissue.

Current bone tissue engineering, based on three-dimensional scaffolds loaded with stem cells, including MSCs, has some limits related, for example, to the administration of cells, such as rejection reactions, the induction of tumours and the transmission of infections. Moreover, stem cell handling requires strict control programmes and elaborate protocols to standardize the production and storage of large quantities of stem cells.

Moreover, the production of scaffolds loaded with stem cells is relatively complex and problems can be encountered to obtain suitable distribution and adhesion of the stem cells on the scaffold.

Holkar K. et al. “Biomaterials and extracellular vesicles in cell-free therapy for bone repair and regeneration: Future line of treatment in regenerative medicine”—Materialia 12 (2020) 100736, propose, in general terms, the user of 3D printing techniques to produce scaffolds for bone regeneration incorporating extracellular vesicles. However, processes that can be concretely implemented and that give fully satisfactory results are not currently available.

Diomede F. et al. “Three-dimensional printed PLA scaffold and human gingival stem cell-derived extracellular vesicles: a new tool for bone defect repair”, Stem Cell Research & Therapy (2018) 9:104, describe the ability in vitro and in vivo of the effect of regeneration of bone defects of scaffolds made of polylactide (PLA) printed in 3D and coated with polyethyleneimine (PEI), enriched with human gingival MSC (hGMSC). In substance, this paper proposes first producing the scaffold and then coating it. However, techniques of this kind can compromise the homogeneous distribution of the coating, above all in its inner nucleus and above all when the scaffold has complex geometry and/or high porosity. Moreover, it is not generally possible to obtain control over the release of active substances, normally leading to a relatively rapid release that can potentially limit the desired efficacy in the medium and long term.

Chen P. et al. “Desktop-stereolithography 3D printing of a radially oriented extracellular matrix/mesenchymal stem cell exosome bioink for osteochondral defect regeneration”, Theranostics 2019, Vol. 9, Issue 9, describe the use of hydrogels printed in 3D and incorporating MSC-derived exosomes for cartilage regeneration. In particular, a desktop-stereolithography (SLA) 3D printing technique is applied using a bioink composed of MSC-derived exosomes, extracellular matrix, decellularized cartilage and gelatin methacrylate (GelMA) hydrogel. However, this bioink does not guarantee sufficient mechanical stability to hold the mechanical stimuli required for bone repair.

### SUBJECT OF THE INVENTION

An object of the present invention is to provide a scaffold for tissue regeneration, particularly for bone tissue regeneration, and the manufacturing method thereof, that overcome the drawbacks described in the prior art.

In particular, an object of the invention is to provide a scaffold that allows improved and controlled reconstruction of the damaged bone tissue. A further object of the invention is to provide a particularly effective, simple and inexpensive method for manufacturing said scaffold.

A further object of the invention is to produce scaffolds produced specifically to allow rapid or slow tissue regeneration, particularly of the damaged bone tissue, according to needs.

In accordance with these objects, the present invention thus relates to a scaffold for tissue regeneration, particularly for bone tissue regeneration, and the manufacturing method thereof as defined in basic terms in the appended claims 1 and 11, respectively.

Further preferred features of the invention are indicated in the dependent claims.

The scaffold of the invention is particularly advantageous as the scaffold, for example made of polymeric material, is loaded (and hence functionalized) with specific cell-derived materials, such as secretome and precisely lyosecretome, i.e., secretome in lyophilized form, in particular mesenchymal stem cell secretome (MSC). In fact, by loading scaffolds with secretome it is possible to produce a bone substitute with improved osteoinductive capacity and without the critical aspects connected to the use of stem cells, as instead occurs in conventional engineered tissues. Moreover, compared to stem cells, secretome, in particular in the form of lyosecretome, is able to be prepared in advance to be ready for use in the case of acute diseases that require prompt treatment.

Other features of the invention, implemented in the preferred embodiments, are also particularly advantageous compared to conventional engineered tissues that combine stem cells and scaffolds.

In particular, it is possible to control the timing of bone regeneration based on clinical needs. In fact, in accordance with the present invention, it is possible to produce scaffolds loaded with secretome having different release capacities, allowing the controlled release of substances, such as lipids and proteins, transported by the secretome, stimulating a controlled osteogenic response in the damaged bone segment temporarily occupied by the system of the invention. Based on the manufacturing method of the system of the present invention, it is possible to obtain a rapid release of lipids and of proteins transported by the secretome and, accordingly, to promote rapid regeneration of the bone tissue, or to promote slow release of said substances with consequent slow bone regeneration. In detail, the choice of the manufacturing method of the system of the invention entails the presence or absence of intermediary compounds, such as alginate and fibroin hydrogel and protamine, which influence the release mechanism and kinetics of the substances involved in bone regeneration.

Moreover, it has been verified that the release speed of proteins and lipids from the scaffold can be further controlled by varying the geometry of the scaffold.

Although the use of 3D bio-printing techniques to build scaffolds loaded with vesicles of mesenchymal stem cells has already been proposed, albeit only in general terms, the present invention allows the concrete implementation of these techniques with high efficiency and completely satisfactory results, above all in terms of controlling release of the active substances.

In accordance with the invention, the scaffold is loaded with lyosecretome comprising both the soluble fraction (proteins) and the insoluble particulate fraction (extracellular vesicles); moreover, the process methods of the invention not only allow the scaffold to be loaded with lyosecretome in an entirely efficient manner, but also ensures accurate control of lyosecretome in use.

In particular, extremely advantageous results are obtained producing the scaffold by means of the co-printing technique and encapsulating lyosecretome in alginate hydrogel or alginate and fibroin hydrogel.

In fact, co-printing allows the simultaneous controlled deposition of different (bio)materials during production of the scaffold; combining the structural material (in particular, but not necessarily, PCL), which guarantees the structural stability of the scaffold and ensures that it can support the mechanical loads required once implanted, and lyosecretome encapsulated in the alginate or alginate and fibroin, a fully functional product is obtained both from a structural point of view and from the point of view of efficacy of release of the active substances.

The properties of shape and porosity of the structural material (for example PCL), combined with the alginate composition incorporating lyosecretome and the associated cross-linking, increase the degree of freedom to control lyosecretome release.

The approach of the invention therefore differs from those proposed in the literature and described previously.

The secretome used to functionalize the scaffold can also be used advantageously to convey other active substances (drugs or the like), in particular incorporated in vesicles or exosomes included in the secretome.

Therefore, in certain embodiments the secretome loaded on the scaffold includes a biologically active substance loaded in the vesicles and/or exosomes of the secretome, or in nanoparticles comprised in the vesicles and/or exosomes of the secretome; for example, the biologically active substance is selected in the group consisting of cardiovascular drugs, drugs of the central and peripheral nervous system, pain medication, antimicrobial drugs, chemotherapeutic agents, vaccines, hormones, vitamins, plant extracts, and phytocomplexes.

### BRIEF DESCRIPTION OF THE DRAWINGS

Further features and advantages of the present invention will be apparent from the description of the following non-limiting embodiments, with reference to the figures of the accompanying drawings, wherein:

FIGS. 1A and 1B are two images acquired by means of Scanning Electron Microscope (SEM) of a scaffold produced in accordance with the invention through 3D printing of a structural material (PCL) and subsequent loading of functional material (secretome) by adsorption, sectioned along two planes orthogonal to each other;

FIG. 2 shows a series of images acquired by means of SEM and in increasing magnifications of a detail of the scaffold of FIG. 1A;

FIGS. 3A and 3B are images acquired by means of Scanning Electron Microscope (SEM) of respective scaffolds produced in accordance with the invention through 3D co-printing of structural material (PCL) and functional material (lyosecretome, contained in alginate hydrogel), sectioned along two planes orthogonal to each other and in increasing magnifications;

FIG. 4 is a graph representing the release kinetics of the functional substances (proteins and lipids) from the scaffold of FIGS. 1A, 1B;

FIGS. 5A and 5B are graphs of release of the functional substances (proteins and lipids) from the scaffolds of FIGS. 3A, 3B;

FIGS. 6A and 6B are graphs of release of functional substances (proteins and lipids) from scaffolds produced in accordance with various embodiments of the invention;

FIG. 7 is a graph of release of proteins from scaffolds produced in accordance with an embodiment of the invention in which fibroin (lyosecretome encapsulated in an alginate and fibroin hydrogel) has been added to the functional material of the scaffolds of FIGS. 3A and 3B.

### PREFERRED EMBODIMENT OF THE INVENTION

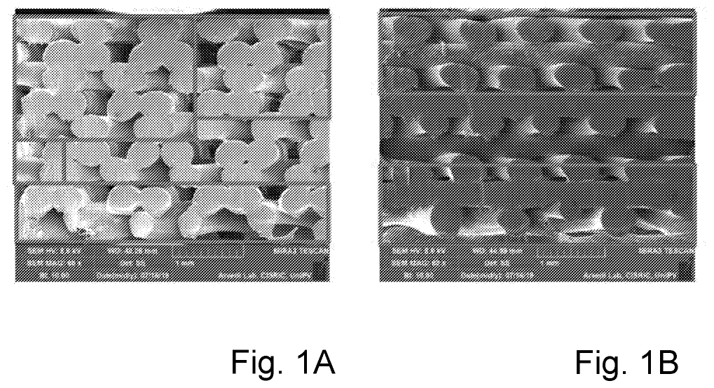

The images of the FIGS. 1A, 1B show a scaffold for tissue regeneration, particularly for bone regeneration, in accordance with the invention.

In the images under the electron microscope of FIGS. 1A, 1B only one portion is shown, sectioned along two vertical planes orthogonal to each other, of the scaffold.

The scaffold comprises a three-dimensional supporting structure produced in a biocompatible structural material; in accordance with the invention, the supporting structure is loaded and hence functionalized with secretome, in particular mesenchymal stem cell secretome, defining a functional material conveyed by the supporting structure.

The supporting structure can have various shapes, sizes and geometries, also according to the specific use for which the scaffold is intended.

In particular, the supporting structure of the scaffold is produced with a biocompatible and optionally bioabsorbable and/or biodegradable polymeric material.

In a preferred embodiment, the supporting structure is made of polycaprolactone (PCL). However, it is understood that other materials can be used, such as polyglycolic acid (PGA), polylactic acid (PLA), poly(lactic-co-glycolic) acid (PLGA), polycaprolactone (PCL), acrylonitrile butadiene styrene (ABS), polycarbonate (PC), polyethylene (PE), poly(methyl methacrylate) (PMMA), other thermoplastic polymers suitable for 3D-printing, mixtures thereof.

In other embodiments the supporting structure is made of bioceramic and/or bioglass, as biomaterials already widely used in the medical field, for example as grafting materials, thanks to their excellent biocompatibility, biofunctionality and mechanical properties.

Alumina (Al₂O₃) is among the most important inert bioceramics for biomedical applications due to its mechanical properties of stiffness and hardness and its properties of chemical resistance to almost all environments.

Bioglasses belong to the class of active bioceramics and are capable of promoting positive reactions of the biological environment to the implant. Specifically, bioglass is a blend of sodium, silicon, calcium and phosphorus oxides.

Analogously to polymeric materials, bioceramic material and bioglass are also suitable for 3D printing.

The supporting structure made of PCL (or other material) is produced through a 3D printing process, for example by means of a 3D bio-printer provided with a pneumatic extrusion system.

The secretome present on the supporting structure is in particular mesenchymal stem cell secretome (MSC-secretome).

Advantageously, MSC-secretome is isolated and purified from supernatants of mesenchymal stem cells (MSCs) in culture by means of a combined ultrafiltration and lyophilization process described in the patent application WO2018/078524.

In brief, the process for obtaining secretome includes the steps of:
(i) collecting from mesenchymal stem cells in culture a supernatant, comprising both a soluble fraction (essentially proteins) and an insoluble fraction in particulate form (vesicles),
(ii) dialyzing or ultrafiltering the biological fluid using a membrane with a cut-off value equal to or lower than 500,000 Dalton and a dialysis or ultrafiltration fluid,
(iii) adding a cryo-protector to the dialyzed or ultrafiltered solution obtained, and
(iv) lyophilizing the resulting solution.

A cryo-protector, preferably mannitol, is added to the ultrafiltered supernatant, in order to prevent undesired effects, such as disintegration of the extracellular vesicles and instability of proteins, caused by rapid freezing and by drying during the lyophilization process. A lyophilized powder, called lyosecretome (lyophilized secretome), which contains the secretome obtained from mesenchymal stem cells (MSCs), is obtained at the end of the process.

In a first embodiment, lyosecretome is loaded onto the supporting structure by immersion of the supporting structure in a solution containing lyosecretome, and subsequent lyophilization, so that the surfaces of the supporting structure are coated with lyosecretome. Specifically, two excipients, such as Lutrol® F127 (poloxamer 407) and NaCl, are added to the solution of lyosecretome. Poloxamer 407 is used to increase the wettability of the supporting structure and facilitate the entry of the solution of lyosecretome into the inner pores of the supporting structure so that loading of lyosecretome is homogeneous. Besides facilitating homogeneous loading of lyosecretome, just as mannitol, poloxamer 407 stabilizes proteins contained in the secretome, preventing its denaturation. On the other hand, the addition of NaCl prevents crystallization of the mannitol on the surface of the supporting structure during the lyophilization process.

In a second embodiment of the invention, lyosecretome is loaded onto the supporting structure directly during the step of making the supporting structure, again by means of a 3D printing process.

The 3D printer is provided with two print-extruders fed by respective print cartridges: a first cartridge contains the polymer (for example, PCL), and the other contains lyosecretome, preferably encapsulated in a hydrogel, for example an alginate hydrogel.

The scaffold functionalized with secretome is then produced by depositing the material of the supporting structure (for example PCL) and the hydrogel containing lyosecretome (for example alginate hydrogel) in a single 3D co-printing process.

In a variant of the 3D co-printing process, a hydrogel, in particular an alginate hydrogel, further containing protamine as cross-linking agent, is used.

In a further variant of the 3D co-printing process, alginate and fibroin hydrogel are used.

### Examples

Some experimental examples that describe the manufacture and efficacy of scaffolds in accordance with the invention are set down below.

1. Preparation of Lyosecretome

Lyosecretome (lyophilized secretome of mesenchymal stem cells) was prepared according to the methods indicated above, substantially as reported in WO2018/078524.

In particular, lyosecretome obtained from adipose-derived cells (AD-MSC) collected from adipose tissue was used. The cells were cultured with standard methods. Secretome release from the cells was obtained culturing the cells in DMEM/F12 without platelet lysate for 48 h; the conditioned media were collected after 9, 24, 33 and 48 hours and grouped together. The MSCs were detached with trypsin-EDTA and tested to assess the cell viability and concordance with all the requirements necessary for clinical use.

The conditioned media were centrifuged at 3500 g/min for 10 min to eliminate cell debris and apoptotic bodies, obtaining supernatants that were collected and ultrafiltered by means of tangential flow filtration. Both the free soluble proteins and the vesicles produced by the MSCs were maintained.

The samples were initially concentrated at 0.5×10⁶ cell equivalents per mL (calculated dividing the total number of cells and the mL of concentrated and purified supernatant) and then diafiltered using sterilized ultrapure water.

Mannitol (final concentration 0.5% w/v) was dissolved in the concentrated and purified secretome; the resulting solution was frozen at −80° C. and lyophilized at 8×10⁻¹ mbar and −50° C. for 72 h.

The lyosecretome obtained was stored at −20° C. until use. Each mg of lyosecretome corresponds to 0.1×10⁶ cell equivalents (calculated dividing the total number of cells used for production and the milligrams of lyosecretome obtained).

2. Preparation of Scaffolds Functionalized with Lyosecretome

Scaffolds functionalized with lyosecretome were prepared both by means of 3D printing of the supporting structure and subsequent adsorption of lyosecretome, and by means of 3D co-printing of the material of the supporting structure (PCL) and of lyosecretome (suspended in a hydrogel loaded with alginate), using two different concentrations of alginate (6% and 10% w/v); an alginate hydrogel cross-linked with a solution at 2% (w/v) of CaCl₂ was used.

2.1. 3D Printing and Subsequent Adsorption of Lyosecretome

Different supporting structures made of PCL, of various shapes and sizes, were printed using a commercial 3D printer. In particular, structures with a cylindrical and parallelepiped shape, of various sizes, were printed. Further samples were printed with other materials selected from: polyglycolic acid (PGA), polylactic acid (PLA), poly(lactic-co-glycolic) acid (PLGA), polycaprolactone (PCL), acrylonitrile butadiene styrene (ABS), polycarbonate (PC), polyethylene (PE), poly(methyl methacrylate) (PMMA), other thermoplastic polymers suitable for 3D-printing, mixtures thereof.

The supporting structures printed in PCL were then immersed in an aqueous solution of lyosecretome (15% w/v), Lutrol® F127 (0.1% w/v) and NaCl (0.1% w/v) for 1.5 h at 4° C., frozen at −80° C. and lyophilized at 8×10⁻¹ mbar and −50° C. for 72 h. Lutrol® F127 was added to increase the wettability of the supporting structure and thus allow more homogenous loading of lyosecretome, while NaCl was added to reduce mannitol crystallization during the lyophilization process.

To determine loading, the scaffolds were dispersed in deionized water under magnetic stirring for 96 h. The solutions obtained were tested to determine the protein and lipid content, obtaining a protein content of 720.29±118.418 μg and a lipid content of 50.10±20.925 μg (mean value±standard deviation obtained from three independent replicas starting from the same batch of lyosecretome).

The considerable variability (high standard deviation) of the protein and lipid content may be indicative of a secretome distribution that is not completely homogenous.

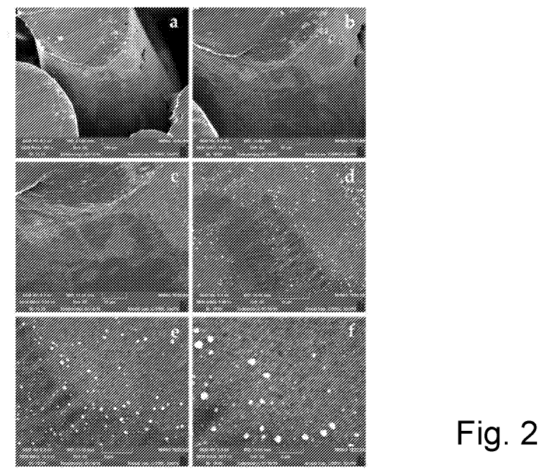

FIGS. 1A, 1B and 2 show the morphological and structural characterization (by means of electron microscope images) of a sample of scaffold produced with the methods indicated. FIGS. 1A and 1B are two vertical sections along respective planes orthogonal to each other; images a-f of FIG. 2 show a detail with increasing magnification and reveal the materials deposited on the surface of the supporting structure made of PCL, in particular proteins and lipids of lyosecretome (a, b, c, d) and NaCl, mannitol and vesicular components of the secretome (e, f).

2.2. 3D Co-Printing

Different scaffolds functionalized with lyosecretome, of various shapes and sizes, were produced by means of 3D co-printing of PCL and an alginate hydrogel incorporating lyosecretome. In particular, porous structures made of PCL were produced, inside which cavities filled with alginate loaded with lyosecretome were obtained.

A 3D bio-printer with a pair of print-extruders was used: the first to extrude the PCL, the second to extrude the alginate. Lyosecretome powder was first dispersed in a solution of alginate with a concentration of 12.5 mg/mL.

Further tests were conducted with other materials, selected from polyglycolic acid (PGA), polylactic acid (PLA), poly(lactic-co-glycolic) acid (PLGA), polycaprolactone (PCL), acrylonitrile butadiene styrene (ABS), polycarbonate (PC), polyethylene (PE), poly(methyl methacrylate) (PMMA), other thermoplastic polymers suitable for 3D-printing, mixtures thereof.

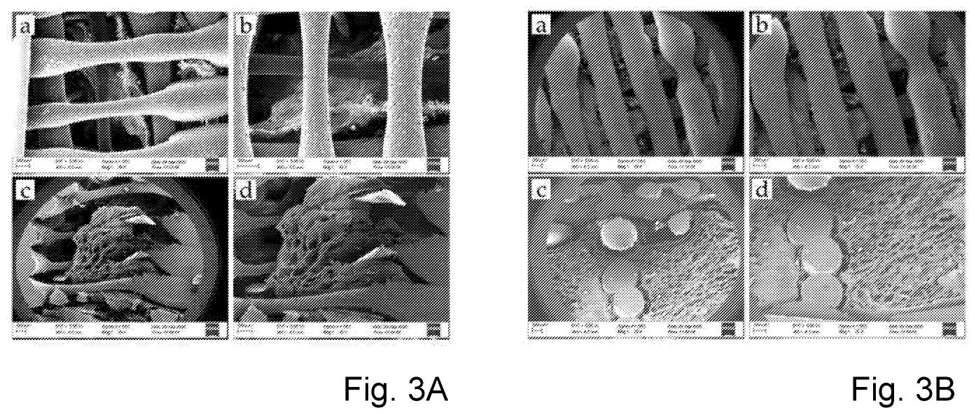

FIGS. 3A, 3B show the morphological and structural characterization (by means of electron microscope images of a portion of scaffold along two vertical sections orthogonal to each other and with increasing magnification) of a sample of scaffold produced with these methods.

Scaffolds were produced with two different alginate concentrations: 6% and 10% (w/v). Both alginate solutions were cross-linked with a solution of CaCl₂ at 2% (w/v).

In some samples, protamine was added to the alginate solution at 10% (w/v) in order to cross-link the alginate with the protamine.

To determine loading, scaffolds were dispersed in deionized water under magnetic stirring for 240 hours. The solutions obtained were tested to determine the protein and lipid content.

For the samples with 6% alginate concentration (PCL-Alg6) the mean protein and lipid content was 188.68±18.386 μg and 25.00±7.042 μg, respectively.

For the samples with 10% alginate concentration (PCL-Alg10), the mean protein and lipid content was 104.52±16.977 μg and 55.10±11.588 μg, respectively. For the samples with 10% alginate concentration and with cross-linking with protamine (PCL-Alg10p), the mean protein and lipid content was 538.63±11.729 μg and 188.68±18.386, respectively.

Further samples were produced with the same methods indicated above and by means of 3D co-printing of PCL and alginate loaded with lyosecretome, varying the geometrical shape of the scaffold, in particular changing from parallelepiped shapes to cylindrical shapes.

The mean lipid and protein content in cylindrical scaffolds with 6% alginate concentration (cPCL-Alg6) was 218.75±243.383 μg and 69.82±76.169 μg, respectively.

The mean lipid and protein content in cylindrical scaffolds with 10% alginate concentration (cPCL-Alg10) was 342.66±39.920 μg and 85.77±0.0521 μg, respectively.

The mean lipid and protein content in cylindrical scaffolds with 10% alginate concentration and cross-linking with protamine (cPCL-Alg10-p) was 543.97±105.892 μg and 37.86±28.954, respectively.

2.2.1 3D Co-Printing with Alginate and Fibroin Hydrogel

Further scaffolds, for example with a cylindrical shape, were produced with the same methods indicated above and by means of 3D co-printing of PCL and lyosecretome incorporated in an alginate and fibroin hydrogel.

In an example, the alginate and fibroin (Alg-Fib) based hydrogel was produced by adding silk fibroin at 5% (w/v) to the alginate solution at 10% (w/v). The Alg-Fib hydrogel was then cross-linked with a solution of CaCl₂ at 2% (w/v) and protamine at 5% (w/v).

The scaffold thus produced was then immersed in KCl at 20% (w/v) for 5 minutes so as to cause transition of the fibroin of the silk from the silk structure I (soluble) to the silk structure II (insoluble).

The fibroin of the silk was extracted from Bombyx mori cocoons according to known procedures described in the following documents: Rockwood D. N. et al. “Materials fabrication from Bombyx mori silk fibroin”, Nat. Protoc. 2011, 6, 1612-1631; Phillips D. M. et al. “Dissolution and regeneration of Bombyx mori silk fibroin using ionic liquids”, J. Am. Chem. Soc. 2004, 126, 14350-14351, to which some variations were made.

In brief, extraction of fibroin from Bombyx mori cocoons involves, in an initial step, cutting the cocoons into 1×1 cm pieces. Subsequently, the pieces of cocoons were degummed in sodium carbonate (Na₂CO₃) 0.02 M for a time ranging from 1 to 4 hours. Based on the duration of degumming, three samples were obtained: one obtained with degumming of 1 hour, one obtained with degumming of 2 hours and one obtained with degumming of 4 hours. After degumming, the fibres were washed with distilled water at 37° C. and dried at room temperature. The fibroin fibres were then solubilized using an aqueous solution of LiBr 9.3 M at 60° C. for 4 hours. The solution obtained was dialyzed (molecular cut-off of 3-5 kDa) against distilled water at room temperature for 72 hours, diluted at 5% w/v and used to prepare the Alg-Fib hydrogel that, after adding lyosecretome, in inserted into one of the two extruders of a 3D printer.

In an example, the preparation process of the Alg-Fib hydrogel incorporating lyosecretome includes the step of:
(a) dissolving the alginate powder in the fibroin solution and mixing until complete homogenization,
(b) pasteurizing the Alg-Fib hydrogel at 75° C. for 1 hour in view of application in conditions of sterility, for example with cells,
(c) dissolving lyosecretome in the Alg-Fib hydrogel.

3. Release Kinetics

The samples of scaffolds were tested by means of immersion in phosphate buffered saline (PBS) at pH 7.2 to assess the release speed of the functional substances, in particular proteins and lipids, incorporated in lyosecretome.

It was observed that the manufacturing method of the scaffold, the geometry of the scaffold and the intermediary compounds (for example alginate hydrogel, fibroin and protamine) all influence the release kinetics.

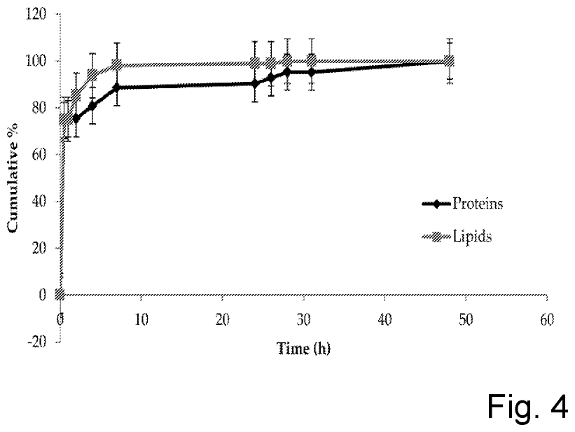

Scaffolds obtained by immersion of supporting structures printed in PCL in a lyosecretome solution have proved to be capable of promoting a relatively rapid release of lipids and proteins from the scaffold. Specifically, scaffolds of this type had released 75% of proteins and of lipids after 30 minutes, 90% of proteins in 24 hours and 98% of lipids in 24 hours (FIG. 4).

On the other hand, scaffolds obtained by 3D co-printing of PCL and alginate hydrogel containing lyosecretome have proved capable of promoting a relatively slow release of lipids and proteins. The slower release kinetics is presumably attributable in this case to the presence of alginate, which acts as diffusion barrier: the greater the alginate concentration, the slower the release of proteins by diffusion is. In fact, in scaffolds having low alginate concentrations (such as PCL-Alg6) a more rapid release of proteins by diffusion was observed compared to scaffolds having higher concentrations of alginate (PCL-Alg10).

On the other hand, the limiting factor related to lipid release is linked to their hydrophobic nature: lipid diffusion is obstructed by the alginate hydrogel based hydrophilic environment.

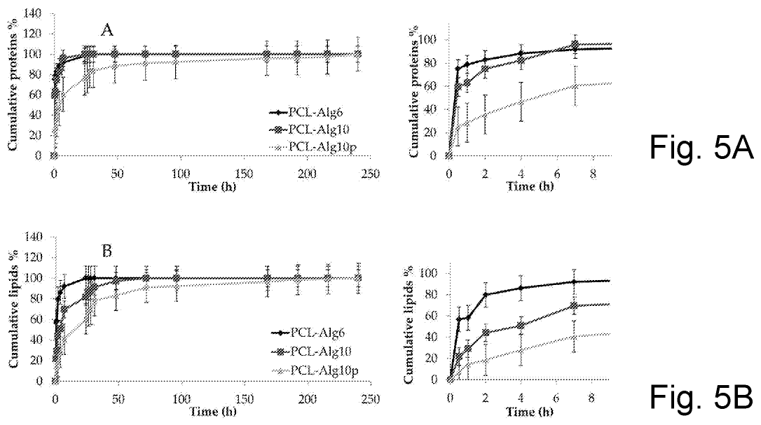

In detail, the following experimental data were obtained (FIGS. 5A, 5B):
the sample PCL-Alg6 released 75% of proteins in 10 minutes and 98% of proteins in 24 hours, while only 57% of lipids was released in 30 minutes, 84% after 4 hours and 100% after 24 hours;
the sample PCL-Alg10 released 60% of proteins after 30 minutes and 100% after 24 hours, while only 21% of lipids was released after 30 minutes, 81% after 24 hours and 100% after 48 hours.

The release kinetics also proved to be slower if an alginate hydrogel cross-linked with protamine was used. In fact, scaffolds produced from PCL-Alg10p released 25% of proteins after 30 minutes and 100% after 240 hours, while only 7% of lipids was released after 30 minutes, 60% after 24 hours and 100% after 240 hours.

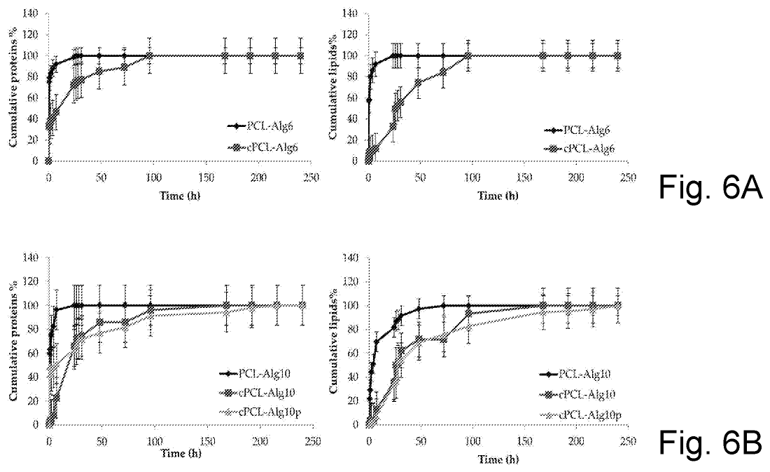

The change in shape from parallelepiped to cylindrical also caused slowing down of the release speed of lipids and of proteins. In fact, as shown in FIGS. 6A, 6B, the cylindrical shaped scaffold (c-PCL-Alg6) released proteins and lipids more slowly compared to the equivalent parallelepiped shaped scaffold (PCL-Alg6): only 32% of proteins were released from the cylindrical scaffold (c-PCL-Alg6) after 30 minutes compared to 75% of the parallelepiped scaffold (PCL-Alg6), while only 5% of lipids were released from the cylindrical scaffold after 30 minutes compared to 57% of the parallelepiped scaffold.

In relation to the scaffolds obtained by means of 3D co-printing of PCL and alginate and fibroin (Alg-Fib) hydrogel incorporating lyosecretome, it was observed how the release of proteins from said scaffolds depends on the degumming time of said fibroin.

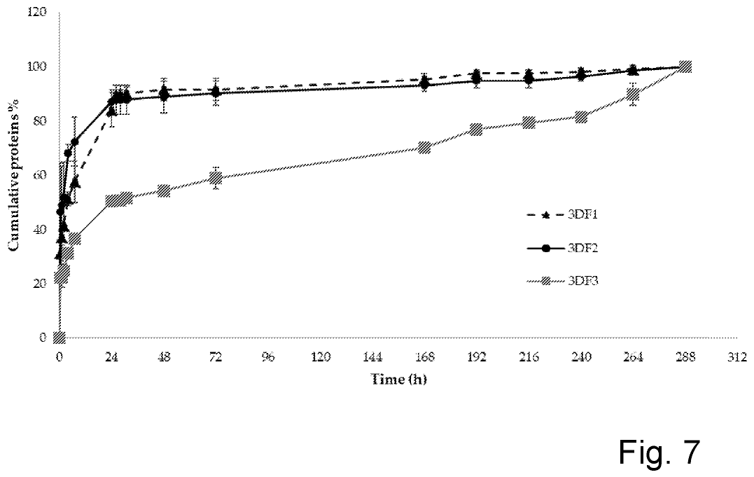

In detail, the following experimental results were obtained (FIG. 7):
the sample 3DF1, in which the fibroin was obtained with degumming of 1 hour, released 31% of proteins in 30 minutes and 84% of proteins in 24 hours;
the sample 3DF2, in which the fibroin was obtained with degumming of 2 hours, released 46% of proteins in 30 minutes and 87% of proteins in 24 hours;
the sample 3DF3, in which the fibroin was obtained with degumming of 4 hours, released 22% of proteins in 30 minutes and 50% of proteins in 24 hours.

It is thus possible to observe that the addition of degummed fibroin for 1 hour (sample 3DF1) or for 2 hours (sample 3DF2) causes a rapid release of proteins from the scaffold. On the contrary, the addition of degummed fibroin for 4 hours causes a slower release of proteins from the scaffold.

Similar results were obtained changing the material of the supporting structure and/or the other parameters identified above.

1. Scaffold for tissue regeneration, particularly for bone regeneration, comprising a supporting structure made of a biocompatible material; characterized in that the supporting structure is functionalized with secretome.

2. Scaffold as claimed in claim 1, wherein the secretome is mesenchymal stem cell secretome.

3. Scaffold as claimed in claim 1, wherein the secretome is lyosecretome, i.e. lyophilized secretome.

4. Scaffold as claimed in claim 1, wherein the secretome includes both a soluble fraction, in particular comprising proteins, and an insoluble particulate fraction, in particular extracellular vesicles.

5. Scaffold as claimed in claim 1, wherein the secretome includes proteins and lipids.

6. Scaffold as claimed in claim 1, wherein the supporting structure is made of a polymeric and/or bioceramic and/or bioglass material.

7. Scaffold as claimed in claim 1, wherein the supporting structure is made of a material selected from: polyglycolic acid (PGA), polylactic acid (PLA), poly(lactic-co-glycolic) acid (PLGA), polycaprolactone (PCL), acrylonitrile butadiene styrene (ABS), polycarbonate (PC), polyethylene (PE), poly(methyl methacrylate) (PMMA), other thermoplastic polymers suitable for 3D-printing, mixtures thereof.

8. Scaffold as claimed in claim 6, wherein the supporting structure contains fibroin.

9. Scaffold as claimed in claim 1, wherein the secretome includes a biologically active substance, which is loaded in vesicles and/or exosomes of the secretome, or in nanoparticles comprised in vesicles and/or exosomes of the secretome.

10. Scaffold as claimed in claim 9, wherein the biologically active substance is selected from the group consisting of cardiovascular drugs, drugs of the central and peripheral nervous system, pain medication, antimicrobial drugs, chemotherapeutic agents, vaccines, hormones, vitamins, plant extracts, and phytocomplexes.

11. Method for manufacturing a scaffold for tissue regeneration, particularly for bone regeneration, as claimed in claim 1, comprising the steps of: making the biocompatible material supporting structure by 3D-printing; and functionalizing the supporting structure with the secretome.

12. Method as claimed in claim 11, wherein the step of functionalizing the supporting structure with the secretome is performed by adsorption of the secretome onto the supporting structure.

13. Method as claimed in claim 12, wherein the secretome is loaded onto the supporting structure by immersion of the supporting structure into a solution containing the secretome, and subsequent lyophilization.

14. Method as claimed in claim 12, wherein the secretome is in solution with poloxamer 407 and/or NaCl.

15. Method as claimed in claim 11, wherein the step of functionalizing the supporting structure with the secretome is performed by 3D co-printing of the biocompatible material of the supporting structure with the secretome.

16. Method as claimed in claim 15, wherein the secretome is loaded onto the supporting structure directly during the step of making the supporting structure, by means of a 3D-printing process with a 3D-printer equipped with a pair of print-extruders fed with the biocompatible material and the secretome, respectively.

17. Method as claimed in claim 16, wherein the secretome is contained in a hydrogel printed by the respective print-extruder.

18. Method as claimed in claim 17, wherein the hydrogel is an alginate hydrogel or an alginate and fibroin hydrogel.

19. Method as claimed in claim 17, wherein the hydrogel contains one or more protamines.
